(19) United States
(12) Patent Application Publication
(10) Pub. No.: US 2022/0378500 A1
(43) Pub. Date: Dec. 1, 2022

(54) DEVICES AND METHODS INVOLVING TRANSMURAL-CAPABLE TISSUE PROCEDURES

(71) Applicant: The Board of Trustees of the Leland Stanford Junior University, Stanford, CA (US)

(72) Inventors: Paul J. Wang, Saratoga, CA (US); Meghedi Babakhanian, Sunland, CA (US)

(21) Appl. No.: 17/772,422

(22) PCT Filed: Nov. 2, 2020
(86) PCT No.: PCT/US2020/058600

§ 371 Date: Apr. 27, 2022

Related U.S. Application Data
(60) Provisional application No. 62/929,640, filed on Nov. 1, 2019.

(51) Int. Cl. A61B 18/14

(52) U.S. Cl. CPC A61B 18/1492; A61B 2018/00577

(57) ABSTRACT

In certain examples, aspects are directed to an ablation tool or other procedure-specific tool to treat or assess biological tissue (e.g., ablate cardiac tissue) having a first tissue side and a second, opposite tissue side at which a magnetic-draw element is to be located. In a specific example, a first magnetic element is associated with or coupled to a catheter tool having an expandable portion to transition from a first state towards a second state for providing an expanded girth, so that the expandable portion surrounds the first magnetic element and moves the procedure-specific tool, in part by the first magnetic element moving via magnetic attraction. While the first magnetic element and the magnetic-draw element align on either side of the biological tissue, the procedure-specific tool may be used for the procedure.

### FEDERALLY-SPONSORED RESEARCH AND DEVELOPMENT

This invention was made with Government support under contract TR003142 awarded by the National Institutes of Health. The Government has certain rights in the invention.

### BACKGROUND

Aspects of the present disclosure are directed to a catheter system useful for tissue-specific applications and/or treatment.

Using atrial fibrillation (AF or AFib) as one of the many problems being addressed by biomedical professionals, AF is perhaps the most common type of irregular heartbeat, and one that affects millions in the USA. AF is associated with significant burden in terms of morbidity and mortality from stroke, heart failure, and impaired quality of life translating to significant effects on healthcare costs and resource use. Despite progress in pharmacologic therapy and multiple catheter design advancements such as tissue contact force and cryoablation, the AF ablation single procedure success rate remains below 50% at one year and less than 20% at five years in patients with persistent AF. On the other hand, the open heart surgery Cox-Maze procedure, is capable of achieving 80-90% success at one year and 70% success at five years by creating lasting surgical incisions. However, almost no patients are willing to undergo such an invasive procedure. Thus, there remains a significant clinical need for patients with persistent AF.

The success of open-heart surgery, however, provides a clear physiological solution to treating persistent atrial fibrillation: to create ablation lesions that are as full-thickness, continuous, and as lasting as surgery without the need for surgery. One of the primary causes of low success rate of catheter ablation is the inability to create these full-thickness ablation lesions.

Despite advances in the field of catheter-base procedures, current catheter-based treatments have limited success rate in patient with longstanding issues (e.g., AF with enlarged atria). In the cardiac field, multiple and other types of procedures may be required with associated higher costs, inconvenience, and separate recovery periods. Moreover, fully transmural (full thickness of tissue) lesions are not guaranteed with current treatments, and this includes AF treatments since the epicardial (outside surface of the heart) and the endocardial (inside surface of the heart) lesions are performed during two separate procedures and may not be well-aligned and/or controlled for interactive manipulation associated with providing better results.

### SUMMARY OF VARIOUS ASPECTS AND EXAMPLES

Various examples/embodiments presented by the present disclosure are directed to issues such as those addressed above and/or others which may become apparent from the following disclosure. For example, some of these disclosed aspects are directed to methods and devices that use or leverage from known techniques of applying energy for ablation and while applicable both to laboratory and other situations, such as applicable to in vivo situations including, among other procedures, ablation to treat AFib.

In one specific example, the present disclosure is directed to a method and/or apparatus (e.g., a system, device or assembly) involving an ablation tool to deliver ablation energy to biological tissue (e.g., cardiac tissue) and/or another procedure-specific tool such as where ablation or another procedure may be performed. With the tissue having a first tissue side, at a second, opposite tissue side, a magnetic-draw element may be located. A first magnetic element is associated with or coupled to a catheter tool having an expandable portion to transition from a first state towards a second state for providing an expanded girth, so that the expandable portion surrounds the first magnetic element and in some instances the tool as well.

In certain specific examples, aspects are directed to an ablation tool or other procedure-specific tool to treat or assess biological tissue having a first tissue side and a second, opposite tissue side at which a magnetic-draw element is to be located. The first magnetic element is associated with or coupled to a catheter tool having an expandable portion to transition from a first state towards a second state for providing an expanded girth, so that the expandable portion surrounds the first magnetic element and moves the procedure-specific tool, in part by the first magnetic element moving via magnetic attraction. While the first magnetic element and the magnetic-draw element align on either side of the biological tissue, the procedure-specific tool may be used for the procedure. In more specific examples, the procedure-specific tool forms is secured to or forms part of the expandable portion as may be the case for ablation procedures, and in other instances, the expandable portion facilitates or defines an area of movement for manipulation of the procedure-specific tool (e.g., freely maneuverable within the area of movement, or secured to the expandable portion with the expandable portion being maneuverable so that the procedure-specific tool as secured to the expandable portion may be appropriately used while the magnetic forces align the catheter tool.

In the specific example of using the expandable portion as an ablation tool, the expanded portion may be configured to act as the ablation element while surrounding the magnetic element. By magnetic attraction, the first magnetic element and the magnetic-draw element align on either side of the biological tissue (e.g., sandwiching the biological tissue which is the subject of the ablation) for administering the procedure.

In certain other examples which may also build on the above-discussed aspects, methods and apparatuses involve configuration of the catheter-based tool to permit passive movement of at least one of the magnetic elements to effect smooth movement of the ablation tool (e.g., including an energy delivering electrode) nearby sensitive or vulnerable tissue areas.

In various related and more-specific embodiments, the expandable portion of the catheter may be in the shape of or include a rolling ball (e.g., nitinol mesh or an elastic balloon). The epicardial catheter can be in a tube shape rail to enclose structures such as pulmonary veins or it can be used for localized ablation. The distal end of the epi-catheter couples to the epicardial tissue through a small vacuum port to maintain the tip of the catheter attached to the tissue. Once these two devices are in proximity of each other, their magnetic attraction permits the ablation lesions to be exactly aligned on the epicardial and the endocardial surface of the heart. Ablation energy is delivered from the epi-side of the heart to create a transmural full thickness lesion. The ablation element is moved gradually from one spot to another within the rail to create the lesion set that mimics a lesion set as used in open-heart surgery. The strength of the magnetic field can be adjusted by altering the proximity of the magnet to the heart surface within the epi-directed rail.

The above discussion is not intended to describe each aspect, embodiment or every implementation of the present disclosure. The figures and detailed description that follow also exemplify various embodiments.

### BRIEF DESCRIPTION OF FIGURES

Various example embodiments, including experimental examples, may be more completely understood in consideration of the following detailed description and in connection with the accompanying drawings, each in accordance with the present disclosure, in which:

FIG. 1 is a diagram of a system, according to certain exemplary aspects of the present disclosure, for carrying out one or more procedures via two catheters as part of the system;

FIG. 2 is a schematic illustration of magnetic attraction and alignment of the two catheters with respect to heart tissue, according to certain exemplary aspects of the present disclosure;

FIG. 3 is diagram of a more-detailed experimental-example system, according to the present disclosure, which illustrates additional aspects in the form of a diagrammatic graph;

FIG. 4A is diagram of a more-detailed example system, according to the present disclosure, involving a balloon embodiment used for an expandable portion which can be associated with a sheath or a catheter;

FIG. 4B is diagram of a more-detailed example system, according to the present disclosure, involving a basket embodiment as an alternative to the balloon embodiment of FIG. 4A; and

FIG. 5 shows an exemplary embodiment of a method of using a transmural ablation system, according to the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of apparatuses, systems and methods involving devices characterized at least in part by interaction of magnetic coupling forces to position a procedural-specific tool such as an ablation tool that is coupled to a catheter having an expandable portion which may expand before the tool is activated and energy is to be delivered from the ablation tool to the tissue. In a particular example, a catheter-secure ablation tool and related aspects are described as being configured for administering a heart-directed ablation for mitigating Atrial Fibrillation (AF or sometimes AFib). While the present disclosure is not necessarily limited to such aspects, an understanding of specific examples in such contexts is provided in the following description to facilitate discussion of example embodiments and related aspects.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same connotation and/or reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination. For information regarding background and of further details regarding related embodiments, experiments and applications which can be combined in varying degrees with the teachings herein, reference may be made to the teachings and underlying references provided in Appendices A, B, and C of the above-referenced U.S. Provisional Application.

Exemplary aspects of the present disclosure involve use of a magnetic-draw element cooperating with a catheter-based apparatus which is associated with an ablation tool. The ablation tool may be configured to deliver ablation energy to biological tissue (e.g., cardiac or other tissue) having a first tissue side and a second, opposite tissue side at which the magnetic-draw element is to be located. In a specific example, adjacent the ablation tool is a first magnetic element also associated with or coupled to the catheter tool. The catheter tool may have an expandable portion, for example, to surround at least the first magnetic element, whereby the expandable portion may transition from a first state to or towards a second state for providing an expanded girth via the second state, so that the expandable portion surrounds the first magnetic element and possibly also the ablation tool unless the expandable portion itself is configured as or to have conductive portions, for example, for acting as the ablation tool. The ablation tool and the first magnetic element are to move, relative to the magnetic draw element (e.g., involving magnetic attraction), so that the first magnetic element and the magnetic-draw element become aligned on either side of the biological tissue for administering the ablation.

Consistent with the above aspects, such a manufactured device or method of such manufacture may involve aspects presented and claimed in U.S. Provisional Patent Application Ser. No. 62/929,640 filed on Nov. 1, 2019 (STFD.417P1), to which priority is claimed and for which the above-referenced figures are largely congruent. To the extent permitted, such subject matter is incorporated by reference in its entirety generally and to the extent that further aspects and examples (such as experimental and/more-detailed embodiments) may be useful to supplement and/or clarify.

Consistent with the above and other aspects of the present disclosure, such apparatus and/or methods may involve simultaneous endocardial and epicardial ablation creating a transmural lesion in vivo. An example is a magnetically coupled two catheter system that can create full-thickness guided ablation across heart tissue with two components of the system being aligned on two sides of the heart. The method can combine two separate procedures into one procedure that enhances alignment for different procedures and for AF patients, and enhances ablation transmurality in AF patients.

In various aspects, a system is directed to transmural tissue ablation characterized by or including an endocardial assembly and an epicardial assembly. The endocardial assembly includes an ablation element, a magnetic element and an expandable element that when expanded surrounds a first magnetic element used for positioning in connection with the ablation. The epicardial assembly includes an ablation element and the first magnetic element that magnetically attracts the magnetic element of the endocardial assembly and allows for alignment of the epicardial assembly with the endocardial assembly on opposite sides of heart tissue. Movement of the epicardial assembly causes passive, smooth movement of the endocardial assembly along an inner surface of the heart.

In a number of embodiments, the endocardial assembly may include an expandable element that when expanded allows the endocardial assembly to move smoothly along endocardial tissue surface. As specific examples, the expandable element may include a balloon or a basket, and/or the endocardial assembly may be configured such that a portion thereof is capable of rotating about the magnetic portion.

Various examples are directed to an apparatus and/or system for transmural tissue ablation characterized by and/or including an endocardial assembly including an ablation element and a magnetic element and an epicardial assembly including an ablation element and a magnetic element. The assemblies may be configured to move together via magnetic force, and create a transmural lesion of tissue when said ablation elements of said assemblies are aligned with each other and sandwich the tissue through said magnetic force. The magnetic force may be adjustable.

In yet further examples and appreciating that while the above example embodiments are discussed in connection with magnets, other mechanisms subject to such attraction and alignment one or more ablation tools (e.g., ablation electrode) or ablation elements in a catheter-based system across the (e.g., heart) tissue are also contemplated; for example, near the end of a catheter and near the first magnetic element, instead of or in addition to such an ablation element, an additional circuit-based sensor and/or proximal sonar element may be secured so as to be aligned for sensing voltage, current and/or impedance, sensing or sending sound, and/or capturing images at the tissue while the first magnetic element and the other magnetic element, to which the first magnetic element is drawn, are aligned. As examples, such treatments and procedures may be used in connection with the following examples (without limitation): ultrasound, impedance/electrical-current sensing circuits, imaging tools (e.g. IR), and internal location-tracking sensors (sometimes referred to as internal GPS sensors).

In connection with other exemplary aspects which may also be applied to one or more of the above examples, apparatuses and methods are directed to one of more of the above aspects and/or features involving applications, without limitation: full-thickness ablation during epicardial and endocardial ablation procedures; treatment of less-than transmural (full tissue) ablation; treatment of non-AF cardiac-related issues; and/or treatment of other ailments of the heart and biological tissue and structure associated with various organs. Apart from cardiac procedures, example uses are directed to the urinary system involving use of a magnetic-drawing element introduced to the outside side of urinary-system tissue (e.g., kidney, ureter and urethra) via a first catheter such as via an endoscopic procedure, and a second catheter having the above-described expandable portion and coupled to or including the first magnetic element may be introduced via the urethra towards and until in sufficient proximity of the magnetic-drawing element at which stage, the magnetic elements become aligned and for sensing and/or detecting (e.g., via imaging or current-based impedance sensing) a differentiating type of structure, tissue, obstruction, etc. By including a controller coupled to one of the catheters (e.g., a logic circuit at the remote end distal from the expandable portion), the magnetic force may be adjusted to accommodate the type of tissue and/or to move the tools up/down/around the targeted area for more accuracy. Such adjustments may be effected, e.g., by movement of the magnetic element(s) to create distance therebetween, by (de-)rotation of one of the magnets so that they are no longer facing each other to effect opposite-polarity draw, and/or by physically or otherwise impeding the magnetic force between the magnetic elements. Once such magnetic-force and/or location-specific adjustment is made, the above procedures may be repeated, for example, based on such movement toward alignment being repeated due to the magnetic force drawing the elements toward one another again. Further, while expanded via the magnetic forces and while alignment of the magnets is in place, the expandable portion of the catheter may be used to define an area for maneuvering the procedurally-specific tool.

In more specific applications, and such example procedures as may be applicable to cardiac, urinary system, colon and other portions of the intestinal tracts, etc., one or two such catheter-based tools may be used in a multi-application process involving a first mode for sensing or detecting targeted tissue and/or structure nearby such tissue (e.g., using one or more of the above sensing mechanisms), and a second mode in which the one or two such catheter-based tools may be used for a further process or treatment of the situation. Whether one, two or more procedural modes, the magnetic-drawing mechanisms may be used with such a procedure-specific tool surrounded by the expandable catheter portion for one or more of (without limitations), and applicable to the targeted tissue area: ablation; application of dye(s) for image-differentiation of aspects of the targeted tissue area; applying radiation for location-specific treatment; obtaining sample tissue for biopsy or detecting/sampling other nearby structure such as cancer cells and even calcium as with kidney-stone structures moving through the system. As a more specific example, these procedures and catheter-based tools may be used in connection with a person's urinary tract releasing blood, where there may be a suspicious protrusion within the ureter (e.g., as detected using conventional noninvasive imaging techniques such as a magnetic resonance imaging and/or x-ray imaging). If the protrusion were to be probed or sampled with conventional tools, the effort may cause puncture and spreading of possibly cancerous cells. By using such sensing tools according to the present disclosure to first assess what cellular structure might be involved, subsequent steps may be taken, whether using the same apparatus or otherwise, for proper treatment or deciding that no treatment is the better option.

In another example, the catheter with the expandable portion (and the first magnetic element) may be applied outside of the narrow canal after the other magnetic-attracting element is already in position. Consider an intestinal blockage in the small intestine. Minimally invasive tools (e.g., camera or dye, etc. accessed through the colon or endoscopic tools) may be used to locate a suspect tissue area inside the small intestine. Once located, the magnetic-attracting element may be positioned. Next, a surgical procedure may involve the catheter with the expandable portion (and the first magnetic element) through the stomach wall to locate where, along twenty-plus feet of small intestine, the magnetic-attracting element was located. Such locating may be conducted via general positioning of the end of the catheter (and/or with camera guidance) until the magnetic forces effect alignment of the magnetic elements. While the expandable portion is expanded, a portion of the expandable portion (or a tool secured or moveably coupled thereto) may be used to examine and/or reposition a target of the small intestine, obtain a biopsy, etc.

Such procedural modes, for example, being used with different tools surrounded by the expandable catheter portion (e.g., with passive and smooth movement attributable to the magnetic forces) may be used to yield significant advantages.

Other particular example embodiments of the present disclosure are directed to administering a procedure, using such an aforementioned system, for assessing and/or treating biological tissue. Consistent therewith, one example method includes: introducing a first assembly, having an procedure-specific tool, a first magnetic element and a catheter, to or towards biological tissue having a first tissue side and a second opposite tissue side at which a magnetic-draw element is to be located, wherein the catheter has an expandable portion to transition from a first state having a first girth to or towards an expanded girth that is greater than the first girth; facilitating movement of the procedure-specific tool and the first magnetic element, relative to the magnetic draw element and by magnetic attraction attributable to the magnetic-draw element, toward an area along the first tissue side at which the magnetic-draw element is to be located; and performing the procedure relative to the biological tissue while the magnetic force is drawing the first magnetic element and the magnetic-draw element toward one another.

In another related more-specific example, the above method may further involve the first magnetic element and the magnetic-draw element becoming physically aligned with one another by way of smooth and passive movement along an inner surface of the tissue to a location for the procedure; for example, with the physical alignment with one another on opposite sides of myocardial tissue of a subject.

In yet another more-specific example, the above method may further involve: inserting the epicardial assembly to a first location relative to the heart tissue; inserting the first (endocardial) assembly to a second location proximal to the first location and the epicardial assembly and on an opposite sides of the heart tissue compared to the first location; causing the expandable portion to transition towards the expanded girth; moving the epicardial assembly smoothly and passively along an inner surface of heart tissue to a location for ablation of the heart tissue, wherein the expandable portion is configured to rotate about an axis relative to the first magnetic element of the first (endocardial) assembly; ablating the heart tissue while the heart tissue is sandwiched between the first magnetic element and the magnetic-draw element.

Such above-described methodologies may further involve adjusting the magnetic force to manipulate the procedural-specific tool, and/or configuring the procedural-specific tool with a sensor and using the sensor to assess the tissue; for example, using the sensor before treatment, after treatment, and/or for various purposes such as to identify or characterize the tissue and to measure the thickness of the tissue.

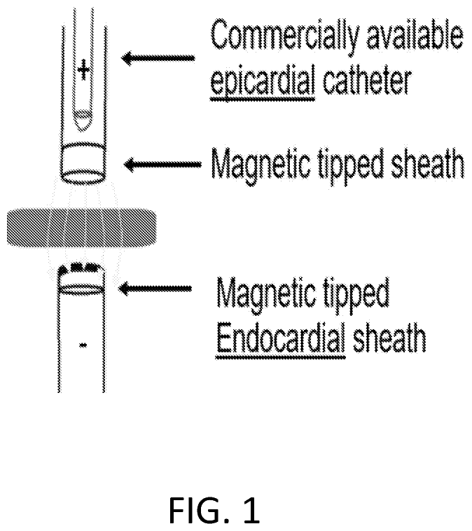

According to other specific examples consistent with the present disclosure, two catheter systems are magnetically enabled so that they can guide the ablation element to the same location. For an ablation application, an epicardial catheter as the procedural-specific tool is designed to guide the ablation element to the epicardial location of interest while the very flexible endocardial catheter follows the element and can reach difficult to access locations. In a specific example, this is unlike current practices in which catheter ablation uses a pad that adheres to patient skin, away from the heart, and acts as the ground pad during ablation. To reduce energy loss and improve transmural lesion formation, according to the present disclosure, a bipolar radiofrequency ablation system is designed to reach across the tissue with the endocardial catheter as the ground pole while magnetically attracted to and aligned with the epi-ablation element (see FIG. 1).

In more specific examples of this (FIG. 1) type, the ablation element(s) may use a ground pole that can be either the epicardial or the endocardial elements, a vacuum or other methodology may be used to couple to the tissue of interest, and as a guiding element, the ablation pattern can be a rail for a path ablation, or a line for a line ablation or a single spot ablation. The ablation element can be a tube-like structure encapsulating or covering the magnet and the ablation element, or it can be a rail that works with a small unit, housing the magnet and the ablation element. Such a small unit may use the rail to follow a path. As a mechanism for attracting and aligning two units across the tissue, magnetic enablement may be used to communicate and align across tissue or any other sensor may be used to monitor location and alignment. In connection with the magnetic enablement and alignment, magnetic strength may be adjusted, for example, through electromagnet or a stepwise hydraulic system on the epicardial side to adjust for magnetic strength and accommodate for the tissue thickness. Optionally, magnetic strength may be monitored through a contact force sensor built into the distal end of the catheter (epi or endo, or both). Magnetic strength could also be detected using other tools such as the Hall-effect sensors or strain or tension sensors, and/or adjusted via electromagnets (e.g., for maintaining constant contact force between the ablation element and the tissue). To maintain a constant magnetic force independent of the tissue thickness, a hydraulic system may be used to adjust the distance or gap between magnetic elements across the tissue based on the magnetic force detected (and can be used for either the endo or epi catheter).

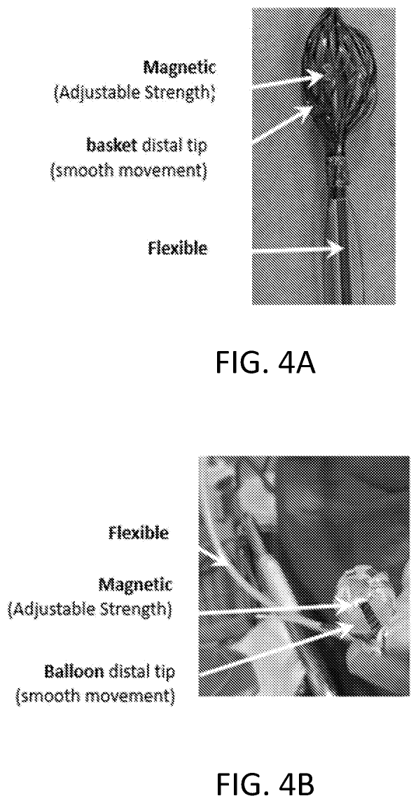

Ablation treatment may be done as bipolar or two unipolar ablation (see FIGS. 4A and 4B as also discussed below).

Experimental/More-Detailed Embodiments

In current practice, catheter ablation uses a pad that adheres to patient skin, away from the heart, and acts as the ground pad during ablation. To reduce energy loss and improve transmural lesion formation, a bipolar radiofrequency ablation system was designed. The system ablates across tissue with an endocardial catheter as a ground pole that may be magnetically attracted to and aligned with an epicardial element or catheter. Certain specific experimental examples in accordance with the present disclosure are directed to a two-catheter system, or two steerable sheaths, that have portions magnetically enabled so that they can guide an ablation element to a desired location of interest in the heart.

FIG. 1 shows an embodiment of an exemplary two catheter system. One catheter can be an epicardial catheter that may be designed to guide one magnetic element and one ablation element to the epicardial side of the location of interest. The second catheter can be a flexible endocardial catheter that may also deliver a magnetic element and an ablation element to the desired location in the heart. The endocardial catheter can follow the epicardial catheter, through magnetic attraction of the magnetic elements, to reach desired and/or difficult to access locations. The endocardial catheter may act as a ground pole for the ablation. Alternatively, the epicardial catheter may serve as a ground pole. The endocardial catheter can have a tip that can be magnetically enabled (magnet/paramagnet) and the design can allow the endocardial catheter to move smoothly along the inter surface of the heart. The design can include an element to allow the distal end of the catheter to roll easily and avoid jumps along the inner surface of the heart.

In some exemplary embodiments, the epicardial catheter can act as a guiding element for one of two ablation elements. Different modalities can be used to perform the ablation including ultrasound, cryoablation, radiofrequency (RF), heating elements, laser, needle, and knife, for example. A desired ablation pattern can be a rail for a path ablation or a line for a line ablation or a single spot ablation. The epicardial catheter can be, for example, (1) a tube encapsulating a magnet and the ablation element, or (2) a rail that works with a small unit, housing the magnet and the ablation element. The small unit may use the rail to follow a path. The tube shape rail may enclose structures such as pulmonary veins or it can be used for localized ablation. Another exemplary design involves use of robotics to drive the ablation unit. One robotic example may be a rover carrying the ablation element around the epicardial surface. The rover may be guided virtually from outside the body or it may be guided using a guided mechanism through thoracoscopy.

In other embodiments, the distal end of the epicardial catheter may couple to the epicardial tissue. One example of a coupling element is a small vacuum port that may maintain the tip of the epicardial catheter attached to the tissue. Other coupling methods/apparatuses are also contemplated. There are other mechanisms that could be used to couple the system to tissue such as magnets across tissue, reverse magnetic strength, balloons or expanded systems to use the space around the heart to press the ablation surface against the heart tissue. Other mechanisms that can be considered include air, tissue glue/polymer, electrical sources, etc.

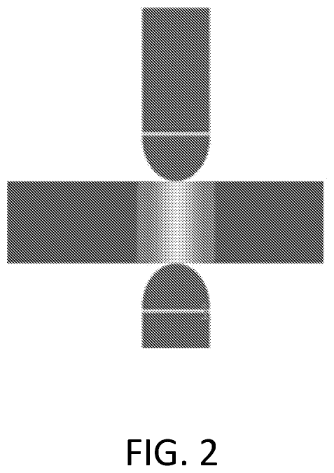

In exemplary embodiments, once the two catheters are in close proximity of each other, and are moved to a desired location in the heart, their magnetic attraction permitting ablation lesions to be exactly aligned on the epicardial and the endocardial surfaces of the heart. FIG. 2 shows a schematic illustration of magnetic attraction and alignment of the two catheters with respect to the heart tissue. Magnetic strength may be supplied through an electromagnet or a stepwise hydraulic system on the epicardial side, for example, to adjust for magnetic strength and accommodate for tissue thickness. Ablation energy may be delivered from the epicardial and endocardial sides of the heart tissue, to create a transmural full thickness lesion. Ablation may be done as bipolar ablation or unipolar ablation. The ablation elements may be moved gradually from one spot to another within a rail or a sheath to create a lesion set that mimics a lesion set used in open heart surgery, for example. The strength of the magnetic field may be adjusted by altering the proximity of the magnet(s) to the heart surface.

Important elements of the proposed system may include a complementary endocardial-epicardial magnetic catheter pair, and the endocardial catheter tip for smooth tracking. The attraction of the magnetic catheters and aligned ablation elements across the heart tissue occurs prior to simultaneous ablation of the tissue from both sides. As an alternative embodiment, rather than the two catheters being magnetically attracted, for example, a sensor or sensors may be used to monitor location and alignment of the catheters.

In exemplary embodiments, the endocardial catheter distal end includes an expandable tip. The design and dimensions of such an expandable element may assist the catheter tip to eliminate crevices in the heart. The expandable tip can be made of an elastic balloon in different shapes, a basket (Nitinol® or other), a combination of a balloon and basket, have an umbrella shape at the tip (endocardial catheter distal end), a spatula shape, or be made of wire mesh with a curvature. The expandable shape, material and design should provide enough support to keep the magnet within aligned in a magnetic-axis and prevent it from bending and being misaligned while ensuring smooth movement of the catheter tip. Magnetic attraction of the magnet in the endocardial catheter to the epicardial catheter magnet allows the magnets to move together in the heart.

In specific embodiments, the distal end of the endocardial catheter, with the expandable element, is able to roll along the inner surface of the heart. Such a rolling feature allows for smooth movement of the catheter tip with certain materials and designs. The expandable tip is designed to roll/pivot around the magnetic-unit's axis.

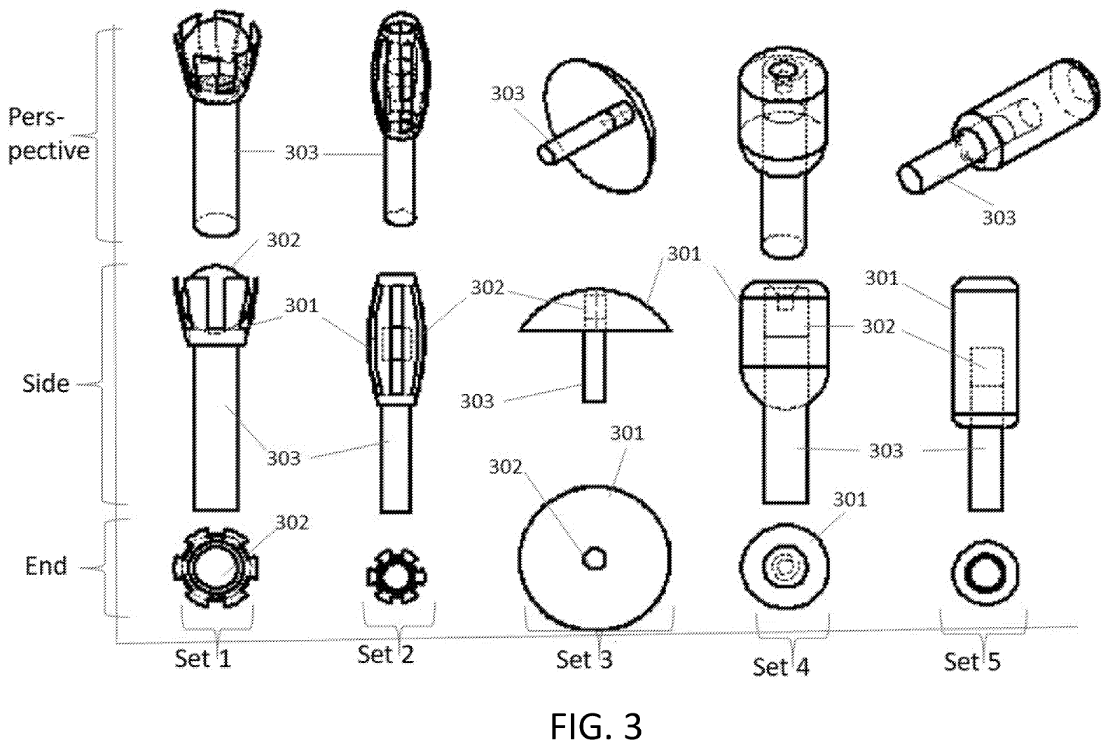

Other designs can include a rotating mechanism (e.g., around an axle) such as a rolling ball inside the catheter distal tip. Graphical depictions of related exemplary devices are shown in FIG. 3, which depicts both a balloon embodiment (as shown in FIG. 4A) and a basket embodiment (as shown in FIG. 4B) of the endocardial tip. The magnetic axes are not indicated, nor are directions of rotation, but they would be understood to be consistent with the movement defined by such an axes. Such embodiments are exemplary and others are also contemplated. More specifically, FIG. 3 depicts an x-y graph, as seen via the y-axis, which shows perspective views, side views and end views (via three respective rows), of five different but related example embodiments which may be used for transmural ablation as discussed above. Via the x-axis, each of these five structures are shown in one of five columns (via the above-noted three perspective views) and identified as view sets (or Sets) 1-5.

Each of the five structures is as described above for application of a procedure relative to biological tissue having a first tissue side and a second, opposite tissue side at which a magnetic-draw element is to be located. The catheter's expandable portion, which may also secure/integrate the procedural-specific tool and/or act as the procedural-specific tool, is identified as 301. The first magnetic element is identified as 302 and serves as one of two cooperative magnetic-force drawing elements, and the catheter (or catheter proximal tube) is identified as 303 in each such example. Further, and for illustrative purposes, with respect to each of these related example transmural-ablation embodiments it can be assumed that at least a portion of the catheter's expandable portion acts as one of the ablation electrodes for the transmural ablation procedure. The magnetic strength axis (not shown) projects from the inside and outwardly towards the other drawing magnet (to be located on the other side of the tissue), and the rotation direction may be around an axis which would be understood to be relative to the first magnet element. The magnetic strength is such that the draw by the second magnetic-draw element on the other side of the tissue keeps the catheter's expandable portion expanded so long as the elements are nearby for alignment, and ground may be defined at a convenient location nearby the catheter's expandable portion.

In the first column (Set 1) of FIG. 3, the perspective view, side view and top-down view are shown for one such catheter-based embodiment having the expandable portion 301 as a basket or balloon or other housing type for permitting a spherical ball to roll inside while stopping the distal end from tilting. Perhaps more apparent from the perspective and side views in the first column, the ball 302 may roll inside the 301 basket/balloon. In certain examples, the ball 302 may be spheric in shape and also made of a magnetic or paramagnetic material (e.g., such as containing iron and being a magnetic or paramagnetic element). The rolling magnetic or paramagnetic element may be used as an endo-distal tip as one of the ablation electrodes. In operation, once the distal tip of the endocardial catheter (draw element) is delivered to the target and the magnetic elements are paired across the tissue, the basket or umbrella or balloon shape tool will expand. This component will have conductive elements embedded, printed or placed on them (e.g., it can be in solid, mesh like, stretchable electronics form) that act as the ablation element when it comes directly in contact with the tissue.

The second column (Set 2) of FIG. 3 has the perspective, side and top-down views shown for another such catheter-based embodiment directed to an expandable portion 301 to act as an ablation tool with a first magnetic element 302 of a diametric or short cylinder shape along an axis inside an expandable portion 301, with the expandable portion 301 as a basket or balloon, or other housing type. Apparent from the perspective and side views, the first magnetic element 302 may be a structure shaped around and/or through a center axis and also made of a magnetic or paramagnetic material as above. The rolling magnetic or paramagnetic element may be used as an endo-distal tip as one of the ablation electrodes. In operation of the example shown in set 2 (and also set 5 below) the basket or balloon acts as a rolling element to ensure smooth movement across the tissue. The rolling basket or balloon or umbrella can rotate around the magnet's axis.

The third column (Set 3) of FIG. 3 has the perspective, side and top-down views shown for another such catheter-based embodiment directed to an expandable umbrella structure 301. The wider umbrella portion of the structure 301 acts as the ablation tool and also as the expandable portion 301 as a basket or balloon or other housing type. The first magnetic element 302 (shaped around and/or through the center axis and also made of a material as above), as with Set 2, may be a diametric or short cylinder shape along and/or under the expandable umbrella portion 301, with the expandable portion 301 as a basket or balloon, or other housing type. In operation of the example shown in set 3 (and also set 4 below), the umbrella and balloon elements act as a cushion to ensure smooth movement.

The fourth column (Set 4) of FIG. 3 has the perspective, side and top-down views shown for another such catheter-based embodiment directed to an expandable draw balloon structure 301 with a magnet 302 inside (which may also made of a material as above). As with Set 3, the wider portion of the structure 301 is expandable and may act as the ablation tool, and may be largely in the shape of a cylinder. In operation, the magnet 302 may be a spherical magnetic or paramagnetic element that rotates inside the half basket or half-balloon shaped holder. The spherical balloon will act as the ablator in this set.

The fifth column (Set 5) of FIG. 3 has the perspective, side and top-down views showing similar structure and functionality as with Set 4, but with an expandable draw balloon structure 301 having a magnet 302 inside and wherein the (outer) balloon structure 301 is to roll or rotate around with the magnet 302.)

In non-ablation examples, one or more of the above catheter-based examples of FIG. 3 may implement the expandable draw balloon structure being secured to another type of procedural-specific tool associated with a different procedure as exemplified in the previous discussion (e.g., a tool for ultrasound, impedance/electrical-current sensing circuits, imaging tools, internal location-tracking sensors, biopsy). A proportional-integral-derivative (PID) feedback controller or other controller connected via the proximal end of the catheter may be used to maneuver the tool, during alignment of the magnetic elements and while the expandable portion is expanded.

Another exemplary aspect of the (e.g., endocardial) catheter tube is that it may have a flexible body. The catheter may have a specific amount of rigidity at the distal tip where the expandable and magnetic elements are located. This feature can allow the catheter to more easily access difficult to reach spots.

In other embodiments, magnetic strength or force between the two magnets in the two catheters may be adjusted through different designs and/or mechanisms. For example, magnetic strength can be monitored through the contact force sensor that are built into the distal end of the catheter (epicardial or endocardial, or both). Magnetic strength can also be detected using other tools such as Hall effect sensors or strain or tension sensors. In another example, in order to maintain a constant magnetic force independent of the tissue thickness, a hydraulic system may be used to adjust the distance or gap between magnetic elements across the tissue based on the magnetic force detected (as can be used for either the endocardial or the epicardial catheter). In yet a further example, another mechanism that can be used to adjust magnetic strength for varying tissue thickness (distance between magnetic elements) is electromagnets. Electromagnets can adjust accordingly to maintain constant contact force between an ablation element and the tissue.

In another exemplary embodiment, an electro/magnetically enabled epicardial-endocardial ablation alignment system capable of pairing ablation elements on two sides of the myocardium is provided. Evaluation of proportional-integral-derivative (PID) feedback controllers for movement manipulation and/or for the purpose of maintaining constant contact force during ablation of variable thickness tissue may be performed. A bench-top magnetic test environment can be constructed for an epicardial-endocardial ablation alignment system using rare neodymium and electro-magnets. A bench-top model may be designed to perform magnetic strength testing across variable thickness. Rare earth neodymium magnets may be used as endocardial system complementary to a fixed or variable electromagnet epicardial system. Force measurement may be performed between the coupled system while the epicardial magnet is guided using an automated control system. A PID controller can be used to maintain force within a predefined range through voltage alteration of electromagnetic strength.

A varying magnetic strength mechanism capable of maintaining constant contact force that can be guided along a path of variable thickness may also be used. PID controlled electromagnetic epicardial catheter has superior performance compared to a fixed magnetic strength system with increased percentage of time within a target force range. The ability to perform tissue thickness characterization via electromagnetic force to manipulate magnetic strength for maintaining constant force on the tissue during ablation is realized. The result is an electromagnetic catheter ablation system with controlled feedback capable of varying magnetic field strength for the purpose of contact force ablation.

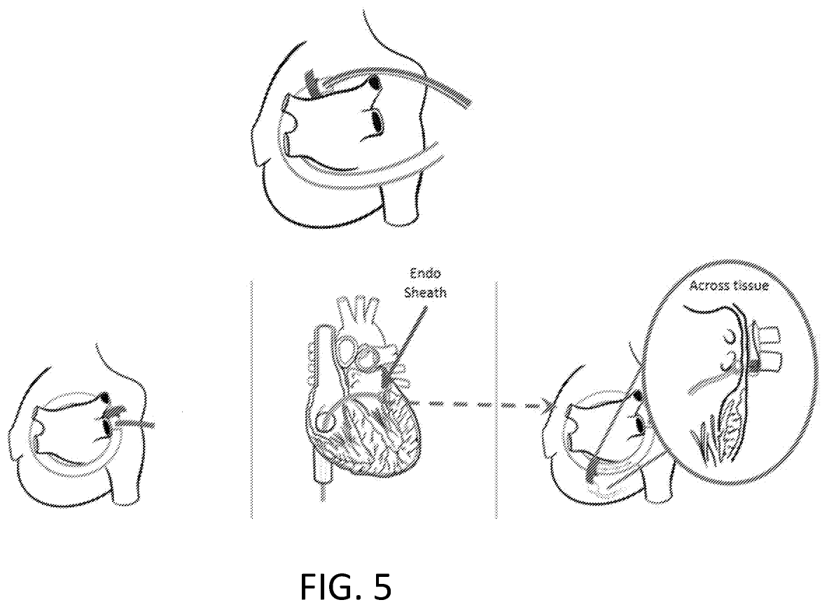

FIG. 5 shows an exemplary embodiment of a method of using the transmural ablation system described herein, with each depiction showing (from left to right) steps one, two and three. As step one on the left of FIG. 5, a flexible epicardial guiding rail may be inserted by first using an introducer tube, such as via standard thoracoscopic approaches. The rail may have a track to guide the epicardial assembly, including an ablation tip and magnet, along a desired ablation path around anatomies. The ablation pattern or path may go around the four pulmonary veins to isolate the pulmonary veins (PVI) or may be in a line or in a single spot, for example.

With reference to the upper depiction of FIG. 5, a guiding rail (lower curved portion in the upper depiction) may be constructed of flexible tubing which has ports for vacuum to gently couple it to the surface of epicardial tissue. The side of the rail opposed against the atrial tissue with the aid of vacuum suction may have an open window for direct contact of the RF epicardial catheter tip with the tissue, while encapsulated inside the tube. Such a design may avoid collateral tissue damage while the catheter follows the rail track through the small space around the heart. The distal end of the epicardial catheter may be coupled to the epicardial tissue through a small vacuum port to maintain the tip of the catheter attached to the tissue.

As step two and shown in the middle of FIG. 5, a flexible endocardial catheter may be introduced through an endo sheath as available in current practice, for example. The endocardial catheter distal tip then may be advanced close to the epicardial element. The distal tip expandable element, which may be a balloon, basket, etc. design, then may be expanded or inflated.

Step three, on the right side of FIG. 5, occurs when the two catheter devices are in proximity of each other. At this time the epicardial catheter may be moved to a desired location, or along a desired path. The endocardial catheter is magnetically attracted to the epicardial catheter through tissue, and also moves accordingly. Their magnetic attraction also permits ablation lesions to be exactly aligned on the epicardial and the endocardial surface of the heart. The thoracoscopic and the catheter sheath can be pulled back. Ablation energy may be delivered from the epicardial side of the heart while the endocardial catheter, for example, may be used as a ground to create a transmural full thickness lesion. The ablation element may be moved gradually from one spot to another within the rail to create a lesion set that mimics the lesion set used in open heart surgery. The strength of the magnetic field may be adjusted by altering the proximity of the magnet to the heart surface within the epicardial rail using the hydraulic mechanism. The moveable epicardial ablation element may measure consistent contact force by adjusting the magnetic strength as necessary depending on the tissue thickness.

In other example embodiments, which may also be used separately or together in combination(s), the rail on the epicardial side or surface can have a train of ablation elements lining up the (entire) rail while touching the tissue. The magnet is guided inside the rail tube which couples the specific element from the endocardial side or surface and performs ablation. Other embodiments can include two rails, one on both the epicardial side or surface and the endocardial side or surface (e.g., across the tissue), to align and perform unipolar or bipolar ablation. In further embodiments and/or in addition, a grid of ablation elements are on the epicardial side or surface and/or the endocardial side or surface. The ablation pattern or spot can be defined and ablation can be induced. The ground pole can be a large surface or a small expandable design that can follow the ablation spot.

Various terms used herein are applicable to contexts as discussed in the disclosure. As examples, the term “ablation element” (aka ablation energy-delivery element) refers to or includes at least one of the following: in the case of RF ablation, electrode(s) such as RF electrodes; and for cryoablation, energy delivery lumen(s), ablation energy return lumen(s), balloon tip such as may be secured to a delivery catheter, and/or other gas/fluid dispenser as may also be secured to a delivery catheter; “magnetic element”, “magnetic mechanism”, “electro-magnetic element”, and “electro-magnetic mechanism” refers to or includes a magnet as structure used for providing the magnetic forces as discussed herein; and the term “element” refers to or includes a structure, and/or may refer to at least one of the following: “portion, “aspect,” “feature,” “component,” “section,” “unit,” “member.” “part,” etc.

It is recognized and appreciated that as specific examples, the above-characterized figures and discussion are provided to help illustrate certain aspects (and advantages in some instances) which may be used in the manufacture of such structures and devices. These structures and devices include the exemplary structures and devices described in connection with each of the figures as well as other devices, as each such described embodiment has one or more related aspects which may be modified and/or combined with the other such devices and examples as described hereinabove.

The skilled artisan would also recognize various terminology as used in the present disclosure by way of their plain meaning. As examples, the Specification may describe and/or illustrates aspects useful for implementing the examples by way of various semiconductor materials/circuits which may be illustrated as or using terms such as layers, blocks, modules, device, system, unit, controller, and/or other circuit-type depictions. Also, in connection with such descriptions, the term “source” may refer to source and/or drain interchangeably in the case of a transistor structure. Such materials (e.g., including portions of conductive or semiconductive structure) and circuit elements and/or related circuitry may be used together with other elements to exemplify how certain examples may be carried out in the form or structures, steps, functions, operations, activities, etc. It would also be appreciated that terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the true spirit and scope of various aspects of the disclosure, including aspects set forth in the claims.

What is claimed:

1. An apparatus comprising:
a procedure-specific tool for application of a procedure relative to biological tissue having a first tissue side and a second, opposite tissue side at which a magnetic-draw element is to be located;
a first magnetic element; and
a catheter coupled to the procedural-specific tool and having an expandable portion to transition from a first state having a first girth to or towards a second state having a second girth that is greater than the first girth, wherein the expandable portion is to surround the first magnetic element and facilitate movement of the procedure-specific tool, relative to the magnetic draw element and by magnetic attraction attributable to a magnetic force involving the magnetic-draw element, toward an area along the first tissue side at which the magnetic-draw element is to be located for administering the procedure via the procedure-specific tool.

2. The apparatus of claim 1, further including a first catheter assembly and a second catheter assembly, wherein the first catheter assembly includes the procedure-specific tool, the first magnetic element and the expandable catheter, and the second catheter assembly includes the magnetic-draw element.

3. The apparatus of claim 1, further including a first catheter assembly and a second catheter assembly, and wherein: the biological tissue includes myocardial tissue between endocardial tissue and epicardial tissue; the first tissue side is part of either the endocardial tissue or the epicardial tissue; the first catheter assembly includes the procedure-specific tool;
the first magnetic element and the expandable catheter, the second catheter assembly includes the magnetic-draw element, one of the procedure-specific tool and the magnetic-draw element is to be located nearest the first tissue side, and the other of the procedure-specific tool and the magnetic-draw element is to be located nearest the second, opposite tissue side; and during a state in which the magnetic-draw element is physically aligned, by magnetically attraction, with the first magnetic element, the procedure-specific tool is to deliver sufficient energy for transmural application relative to the myocardial tissue between endocardial tissue and epicardial tissue.

4. The apparatus of claim 3, wherein movement of the first catheter assembly is to be moved toward a target area of the epicardial tissue-along an inner region or surface of the heart, and wherein the expandable portion refers to and/or is secured to an electrode as the procedural-specific tool for an ablation procedure.

5. The apparatus of claim 1, further including a transmural tissue ablation assembly having the procedure-specific tool, the first magnetic element, and the expandable catheter, wherein at least a section of the expandable portion is configured as the procedure-specific tool, and wherein in response to the expandable portion moving toward or being in the second state, the expandable portion is to surround the first magnetic element and permit the application of the procedural-specific tool as an electrode in a transmural-tissue-ablation procedure.

6. The apparatus of claim 1, wherein the expandable portion is configured to rotate about an axis of the first magnetic element.

7. The apparatus of claim 1, further including a first catheter assembly having the procedure-specific tool, the first magnetic element and the expandable catheter, and wherein the procedure-specific tool is to perform transmural tissue ablation of the biological tissue after and/or in response to the magnetic attraction causing alignment of the first magnetic and the magnetic-draw element and causing the biological tissue to be sandwiched between the first magnetic and the magnetic-draw element.

8. The apparatus of claim 1, further including a controller to adjust the magnetic force and/or cause movement of one of the first magnetic elements and the magnetic-draw element. and wherein the expandable portion further defines an area for manipulative movement of the procedural-specific tool.

9. The apparatus of claim 1, further including a first catheter assembly having the procedure-specific tool, the first magnetic element and the expandable catheter, and wherein in response to or after the transition, the first catheter assembly is configured to move smoothly along endocardial tissue surface.

10. The apparatus of claim 1, further including a first catheter assembly having the procedure-specific tool, the first magnetic element and the expandable catheter, and a second catheter assembly having the magnetic-draw element, wherein the expandable portion of the catheter is to rotate about an axis of the first magnetic element in response to movement of at least one of the assemblies relative to a patient and/or another of the assemblies.

11. The apparatus of claim 1, wherein the expandable portion of the catheter includes a basket and/or a balloon that is to expand.

12. A method of administering a procedure relative to biological tissue, the method comprising:
introducing a first assembly, having an procedure-specific tool, a first magnetic element and a catheter, to or towards biological tissue having a first tissue side and a second, opposite tissue side at which a magnetic-draw element is to be located, wherein the catheter is coupled to the procedural-specific tool and has an expandable portion to transition from a first state having a first girth to or towards an expanded girth that is greater than the first girth;
causing the procedure-specific tool and the first magnetic element to move, relative to the magnetic draw element and by magnetic attraction attributable to a magnetic force involving the magnetic-draw element, toward an area along the first tissue side at which the magnetic-draw element is to be located; and
performing the procedure relative to the biological tissue while the magnetic force is drawing the first magnetic element and the magnetic-draw element toward one another.

13. The method of claim 12, wherein during at least a second state the expandable portion facilitates positioning of the procedure-specific tool and surrounds first magnetic element.

14. The method of claim 12, wherein during at least a portion of a second state the first magnetic element and the magnetic-draw element are physically aligned with one another by way of smooth and passive movement along an inner surface of the tissue to a location for the procedure.

15. The method of claim 12, wherein the magnetic-draw element is part of an endocardial assembly and the first assembly with the first magnetic element magnetically attracting the magnetic-draw element of the endocardial assembly and wherein the first magnetic element and the magnetic-draw element move relative to one another for physical alignment with one another on opposite sides of myocardial tissue of a subject.

16. The method of claim 15, further comprising:
inserting the epicardial assembly to a first location relative to the heart tissue;
inserting the first assembly to a second location proximal to the first location and the epicardial assembly and on an opposite sides of the heart tissue compared to the first location;
causing the expandable portion to transition towards the expanded girth;
moving the epicardial assembly smoothly and passively along an inner surface of heart tissue to a location for ablation of the heart tissue, wherein the expandable portion is configured to rotate about an axis relative to the first magnetic element of the first assembly;
ablating the heart tissue while the heart tissue is sandwiched between the first magnetic element and the magnetic-draw element.

17. The method of claim 12, further including adjusting the magnetic force to manipulate the procedural-specific tool, and wherein the expandable portion further defines an area for manipulative movement of the procedural-specific tool.

18. The method of claim 12, wherein the procedural-specific tool includes a sensor and further including using the sensor to assess the tissue.

19. The method of claim 12, wherein the expandable portion is to surround the first magnetic element and the procedure-specific tool and/or is to expand to surround the first magnetic element and to be used as part of the procedure-specific tool, and to cause the procedure-specific tool to move, relative to the magnetic draw element and by magnetic attraction attributable to a magnetic force involving the magnetic-draw element, toward an area along the first tissue side at which the magnetic-draw element is to be located.

20. The method of claim 12, wherein the procedure specific to the tissue includes assessing or differentiating structure nearby or associated with the tissue, and wherein the procedure-specific tool is used to ablate and create a transmural lesion of the tissue.
